(19) United States
(12) Patent Application Publication
(10) Pub. No.: US 2017/0343551 A1
(43) Pub. Date: Nov. 30, 2017

(54) METHOD OF DIAGNOSING AND TREATING PROSTATE CANCER

(71) Applicants: Hari K. KOUL, Shreveport, LA (US); Fengtian WANG, Shreveport, LA (US)

(72) Inventors: Hari K. KOUL, Shreveport, LA (US); Fengtian WANG, Shreveport, LA (US)

(73) Assignee: Board of Supervisors of Louisiana State University and Agricultural and Mechanical College, Baton Rouge, LA (US)

(21) Appl. No.: 15/479,763

(22) Filed: Apr. 5, 2017

Related U.S. Application Data
(60) Provisional application No. 62/318,467, filed on Apr. 5, 2016.

(51) Int. Cl. G01N 33/574; C12N 15/09; C12Q 1/68; G01N 33/15

(52) U.S. Cl. CPC G01N 33/57434; G01N 33/15; C12N 15/09; C12Q 1/6886; C12Q 2600/158

(57) ABSTRACT

A method of diagnosing a potentially lethal prostate cancer in a patient comprising measuring one of a level of SAM Pointed Domain Containing ETS Transcription Factor (SPDEF) expression in the patient, a level of TWIST1 protein in the patient, and a level of matrix metalloproteinase 9 (MMP-9) expression in the patient, and diagnosing the patient with potentially lethal prostate cancer if one of the level of SPDEF in the patient is lower than a normal level of SPDEF, the level of TWIST1 in the patient is higher than a normal level of TWIST1, and the level of MMP-9 in the patient is higher than a normal level of MMP-9.

### CROSS REFERENCE TO RELATED APPLICATIONS/PRIORITY

The present invention claims priority to U.S. Provisional Patent Application No. 62/318,467 filed Apr. 5, 2016, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND OF THE INVENTION

Prostate cancer (PCa) is the third most commonly diagnosed cancer in the world. Advanced genomic studies have characterized it as a molecularly heterogeneous disease with a multiple clinical spectrum ranging from indolent to highly aggressive. Conventional therapies produce a high rate of cure for patients with localized PCa, but there is no effective treatment for metastatic PCa. These facts underline the urgent, yet unmet, need for identification and characterization of new targets that can help distinguish metastatic PCa from an indolent disease, and pave a way for novel mechanism based anti-metastasis therapies against PCa. Currently available tests for prostate cancer are non-specific. The most common test, PSA test, is a non-specific marker for prostate enlargement. It does not distinguish PCa from other non-malignant prostatic disease. Other methods, DRE/digital rectal exam helps feel the prostate with a figure to see if there are nodules, but this test is also not able to tell which patients have lethal vs. indolent disease. Prostate cancer diagnosis is confirmed by taking tissue biopsies, and grading them based on morphology (Gleason scale/score). Gleason grading is useful in differentiating cancer from non-cancerous morphology, but by and large this test is ineffective in separation a majority of patients that harbor lethal prostate cancer (including those cancers that are likely to cause death within 1, 3, or 5 years from diagnosis) from otherwise indolent disease (including tumors that are unlikely to cause death, including death in 5, 10, or 20 years).

### SUMMARY OF THE INVENTION

Wherefore, it is an object of the present invention to overcome the above mentioned shortcomings and drawbacks associated with the current technology. The present invention is directed to methods and apparatuses that satisfy the above shortcomings and drawbacks. The method and apparatus comprise a method of diagnosing a potentially lethal prostate cancer in a patient comprising measuring one of a level of SAM Pointed Domain Containing ETS Transcription Factor (SPDEF) expression in the patient, a level of TWIST1 protein in the patient, and a level of matrix metalloproteinase 9 (MMP-9) expression in the patient, and diagnosing the patient with potentially lethal prostate cancer if one of the level of SPDEF in the patient is lower than a normal level of SPDEF, the level of TWIST1 in the patient is higher than a normal level of TWIST1, and the level of MMP-9 in the patient is higher than a normal level of MMP-9. According to a further embodiment, the level of SPDEF expression in the patient is measured and the patient is diagnosed with potentially lethal prostate cancer if the level of SPDEF is lower than the normal level. According to a further embodiment, the level of TWIST1 in the patient is measured and the patient is diagnosed with potentially lethal prostate cancer if the level of TWIST1 is higher than the normal level. According to a further embodiment, the level of MMP-9 expression in the patient is measured and the patient is diagnosed with potentially lethal prostate cancer if the level of MMP-9 is higher than the normal level. According to a further embodiment, both the level of SPDEF expression in the patient and the level of TWIST1 in the patient is measured and the patient is diagnosed with potentially lethal prostate cancer if both the level of SPDEF expression is lower than the normal level of SPDEF and the level of TWIST1 is higher than the normal level of TWIST1. According to a further embodiment, both the level of SPDEF expression in the patient and the level of MMP-9 in the patient is measured and the patient is diagnosed with potentially lethal prostate cancer if both the level of SPDEF expression is lower than the normal level of SPDEF and the level of MMP-9 expression is higher than the normal level of MMP-9. According to a further embodiment, both the level of TWIST1 in the patient and the level of MMP-9 in the patient is measured and the patient is diagnosed with potentially lethal prostate cancer if both the level of TWIST1 is higher than the normal level of TWIST1 and the level of MMP-9 expression is higher than the normal level of MMP-9. According to a further embodiment, the level of SPDEF expression in the patient, the level of TWIST1 in the patient, and the level of MMP-9 expression in the patient is measured. According to a further embodiment, the patient is diagnosed with potentially lethal prostate cancer if the level of SPDEF expression is lower than the normal level of SPDEF, the level of TWIST1 is higher than a normal level of TWIST1, and the level of MMP-9 expression is higher than the normal level of MMP-9. According to a further embodiment, the normal level of SPDEF expression is lower if the patient is African American than if the patient is not African American.

The present invention also relates to a method of diagnosing and treating prostate cancer comprising measuring one of a level of SAM Pointed Domain Containing ETS Transcription Factor (SPDEF) expression in the patient, a level of TWIST1 protein in the patient, and a level of matrix metalloproteinase 9 (MMP-9) expression in the patient, diagnosing the patient with potentially lethal prostate cancer if one of the level of SPDEF in the patient is lower than a normal level of SPDEF, the level of TWIST1 in the patient is higher than a normal level of TWIST1, and the level of MMP-9 in the patient is higher than a normal level of MMP-9, and administering a therapeutic to the patient diagnosed with potentially lethal cancer. According to a further embodiment, the therapeutic is a DNA demethylating agent. According to a further embodiment, the therapeutic is one of azacytidine and decitabine. According to a further embodiment, the therapeutic is SPDEF. According to a further embodiment, the patient is human.

The present invention also relates to a kit for diagnosing potentially lethal prostate cancer in a patient comprising an assay for determining one of a level of SAM Pointed Domain Containing ETS Transcription Factor (SPDEF) expression in the patient, a level of TWIST1 protein in the patient, and a level of matrix metalloproteinase 9 (MMP-9) expression in the patient.

The present invention also relates to using protein expression of two specific genes to predict lethal versus indolent PCa.

The present invention also relates to stratifying subsets of prostate cancer patients that will be candidates for DNA demethylating agents (azacytidine and decitabine) as sensitizers for existing therapies.

The inventors have shown that loss of SAM Pointed Domain Containing ETS Transcription Factor (PDEF, a.k.a SPDEF), a putative tumor suppressor, could lead to more invasive phenotype in PCa cell lines through promoting Epithelial to Mesenchymal transition (EMT). The disclosed test is believed to be a first test in its class that can help separate the patients that can have a median survival of 5.2 years vs. a median survival of over 10 years. Thus the disclosed invention can help physicians identify patients that require aggressive therapy and at the same time spare a large number of men from undergoing unnecessary therapeutic interventions. Perhaps identify a group of patients that are more appropriate for active surveillance. The presently disclosed invention will be able to separate subsets of men with prostate cancer that harbor lethal cancer from the ones that have otherwise indolent disease.

Among genes that regulate EMT, Twist1 can inhibit the expression of E-cadherin, increasing tumor motility thus leading to metastasis. The inventors evaluated the role of PDEF and Twist1 in prostate cancer through NCBI gene expression omnibus and The Cancer Genome Atlas (TCGA). The inventors also evaluated the mechanism by which PDEF is regulated epigenetically.

PDEF expression was monitored by immunohistochemistry using PCa tissue microarray. Western blot was employed to measure the PDEF level in PCa cell lines, PDEF and Twist1 gene expression was measured by real time PCR. The PDEF promoter was cloned and methylation specific PCR was performed. PDEF methylation in clinical samples was extracted from TCGA database and PDEF/Twist1 microarray data was extracted from GSE16560 in NCBI gene expression omnibus with R language. GraphPad prism was used to generate figure and statistical analysis. P<0.05 is considered significant.

Immunohistochemistry (IHC) staining of PDEF on PCa patient samples and in commonly used PCa cell lines suggested an inverse correlation between the level of PDEF and Twist1 and in the level of PDEF and prostate cancer progression. Over-expression of PDEF in PC3 cells inhibits Twist1 expression. DNA Methylation status near the promoter region of PDEF was negatively associated with the expression levels of PDEF in established PCa cells. These results suggest that PDEF levels are regulated in part by promoter methylation. Using TOGA data the inventors identified 12 methylation sites and the methylation level of multiple sites correlate with the increased of Gleason Score (GS). Analysis of gene expression data from GSE16560 revealed that low expression of PDEF and high expression of Twist1 were independently associated with decreased or poor survival in PCa patients. Integrated PDEF and Twist1 expression in prostate cancer patient samples distinguished a high-risk group of people for hormone refractory PCa along with short median survival time.

The inventors concluded from the data that loss of PDEF combined with gain of Twist1 expression can serve as a potential biomarker set for distinguishing aggressive PCa from an indolent disease. PDEF expression is apparently epigenetically regulated in part by promoter hyper-methylation and prostate cancer patients with PDEF loss may respond more favorably to DNA de-methylating agents (azacytidine and decitabine) compared to drugs for prostate cancer in clinical practice.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention, The invention will now be described, by way of example, with reference to the accompanying drawings in which:

FIGS. 1A-1E show decreased PDEF expression in aggressive prostate cancer cells. FIG. 1A shows in vitro, PDEF demonstrates differential expression in several prostate tumor cells lines via western blot. FIG. 1B shows PDEF re-expressed in the PC3 cell line. FIG. 1C shows, using shRNA technology, PDEF was knocked down in the LNCaP cell line. FIG. 1D shows, using these cell lines, that PDEF expression regulates tumor cell aggressiveness as demonstrated by decreased cell migration and increased cell migration to LNCaP cells expressing PDEF shRNA. FIG. 1E shows, using these cell lines, that PDEF expression regulates tumor cell aggressiveness as demonstrated by decreased invasion in the PDEF overexpressing PC3 cells, and increased invasion in LNCaP cells expressing PDEF shRNA;

FIG. 2 shows PDEF overexpression inhibits prostate cancer metastasis;

FIG. 3 shows MMP9 mRNA levels are down regulated in response to PDEF expression in vitro and in vivo. Left panels: Representative blots showing mRNA expression of various genes in Vector control PC3 cells or PDEF-PC3 cells and in tumor xenografts. Right panels: Graphs represent densitometry of three independent experiments of the RTPCR data relative to the GAPDH. MMP9 mRNA levels were significantly down regulated in both the cell line and in the subcutaneous tumors over expressing PDEF. (VC-Luc=VC-PC3-Luc and SPDEF OE-Luc=PDEFPC3-Luc) *indicates statistical significance (p<0.05) compared to VC-Luc cells

FIGS. 4A-4E show PDEF overexpression reduced MMP-9 expression and activity and decreased cell invasion. 4A) RTPCR analysis of MMP-9 mRNA expression. 4B) Gelatin Zymography was performed to determine the level of active MMP-9. 4C) MMP-9 promoter activity in PC3 cells overexpressing PDEF as determined by Luciferase reporter assay. 4D-4E) Invasion of PC3 cells expressing PDEF. FIG. 4A shows that PDEF expression completely abolished MMP-9 mRNA expression, as compared to vector control. FIG. 4B shows that PDEF expression completely abolished MMP-9 enzymatic activity, as compared to vector control. FIG. 4C shows that PDEF expression significantly reduced MMP-9 promoter activity, as compared to vector control. FIG. 4D shows that expression of the PDEF significantly inhibited the ability of PC3 cells to invade through Matrigel compared with vector transfected control cells. FIG. 4E also shows that expression of PDEF significantly inhibited the ability of PC3 cells to invade through Matrigel compared with vector transfected control cells.

FIG. 5A is six photographs showing an inverse relationship between PDEF expression and high Gleason grade for prostate cancer.

FIG. 5B is pie charts showing an inverse relationship between PDEF expression and high Gleason grade for prostate cancer;

FIG. 6 shows PDEF expression in prostate cancer tissue sections from Caucasian and African American men;

FIG. 7A is a bar chart showing PDEF down regulates TWIST-1 expression in PC3 cells. FIG. 7B is two pie charts showing low PDEF/highTWIST1 predict aggressive disease in PCa patients. FIG. 7C is three line charts showing low PDEF/highTWIST1 predicts poor survival (7C) in PCa patients.

FIGS. 8A-8C show SPDEF is subjected to DNA methylation regulation. FIG. 8A shows PC3 cells were treated with demethylation agent, 5-aza-2′-deoxycytidine (5-Aza) and the expression of SPDEF mRNA were examined by RT-PCR (A). TMS1, a known methylation-repressed gene, was used as a positive control 5-aza treatment induced the expression of both SPDEF and TMS1. FIG. 8B shows treatment with valproic acid (VPA), a HDAC inhibitor, did not alter the mRNA level of SPDEF. FIG. 8C shows 5-aza treatment reduces cell migration ability of PC3 cells.

FIGS. 9A-9B show SPDEF promoter is differentially methylated in prostate tumor cells. FIG. 9A shows CpG frequency in the SPDEF locus. FIG. 9B shows the methylation status of CpG islands on the proximal promoter of SPDEF in PC3 and LNCaP cells; and

FIGS. 10A-10B show characterization of SPDEF promoter. FIG. 10A shows generation of SDPEF promoter fragments. In order to identify the critical cis-elements and transcription factors that regulate the expression of SPDEF gene, the inventors obtained (by PCR) serial fragments that span different areas of PDEF promoter and cloned them into pGL3. In FIG. 10B, these reporter constructs (SPDEF-Luc) were transfected into LNCaP cells and luciferase activities were measured. Renilla luciferase was used to normalize the transfection efficiency. FIG. 10B shows in silico transcription factor binding site analysis identified putative binding sites for C/EBP-α and YY1 on SPDEF proximal promoter.

### DETAILED DESCRIPTION OF THE PREFERRED EMBODIMENTS

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1, The term “at most” followed by a number is used herein to denote the end of a range ending with that number (which may be a range having 1 or 0 as its lower limit, or a range having no lower limit, depending upon the variable being defined), For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number, For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

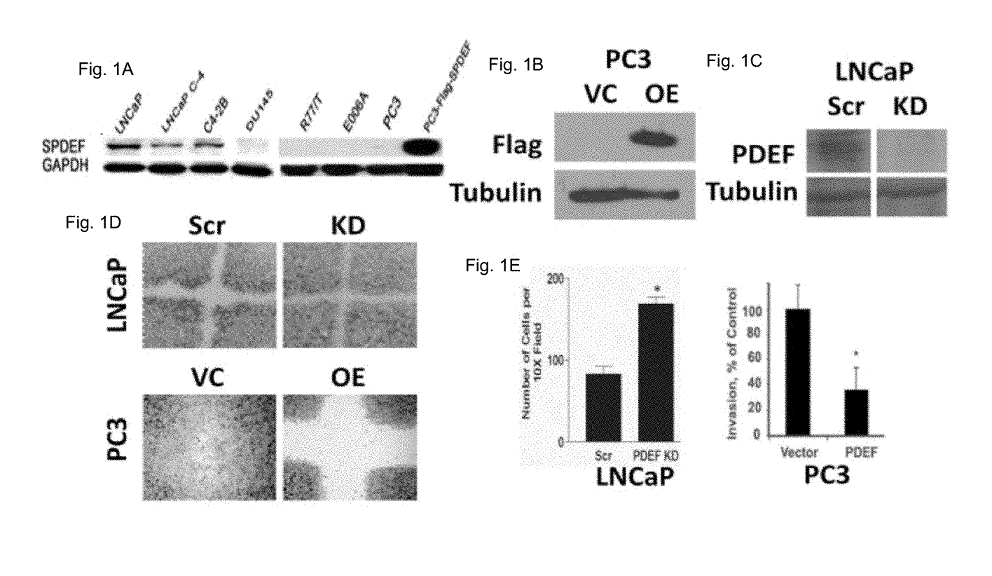

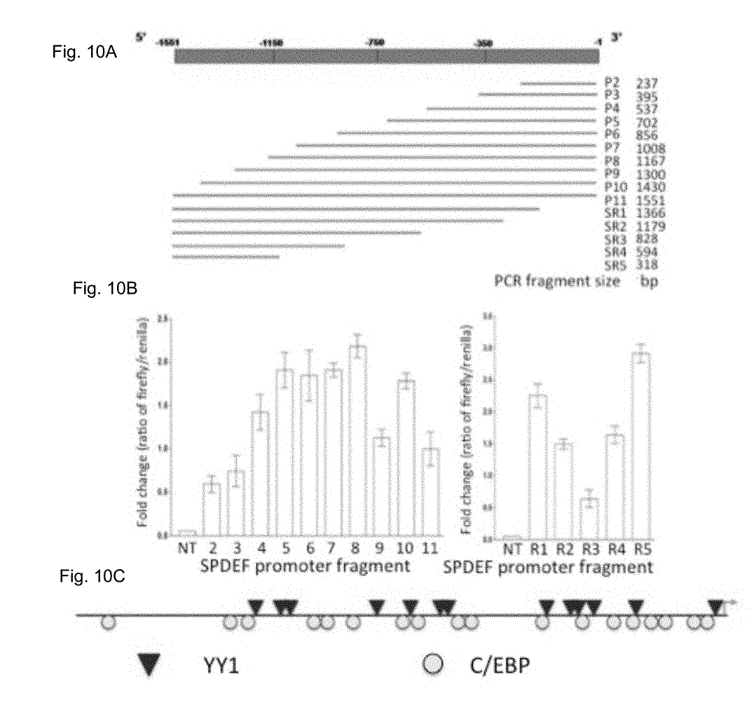

Turning now to FIGS. 1A-10C, a brief description concerning the various components of the present invention will now be briefly discussed.

Progression of prostate cancer from focal, androgen-dependent lesions to androgen-independent, metastatic cancer requires deregulation of growth control, invasiveness and cell motility. ETS (E-twenty-six transformation-specific) are cellular homologues of the viral Ets oncogene of the E26 virus and functions as a sequence specific transcription factors. ETS are involved in a multitude of normal and pathological cellular processes, and play an important role in proliferation, differentiation, development, apoptosis, migration, invasion and angiogenesis. Multiple ETS factors are associated with cancer, such as through gene fusion including prostate cancer. The inventors observe that SAM Pointed Domain Containing ETS Transcription Factor (PDEF, a.k.a SPDEF) appears to play a significant role in tumorigenesis in prostate, breast, colon, and ovarian cancer. PDEF is identified as a prostate-derived ETS factor, present in normal prostate luminal cells. PDEF is unique among ETS factors because of its expression is highly restricted to the tissues with high epithelial content, viz, epithelial cells of prostate, mammary gland, endometrium, and ovary, salivary gland and colon. PDEF is known to the inventors to play a role in regulating cell motility and invasion. The inventors note that PDEF mRNA and protein levels do not always correlate, which may have led to different conclusions in some of the studies examining PDEF expression in primary tumors. To date there are few data available that formally correlate PDEF expression in maintenance of prostate malignant phenotype. Previous studies, reached opposite conclusions with respect to the role of PDEF in prostate cancer. The role of SPDEF in cancer tissues remains debated. Moreover PDEF has never been studied with respect to prostate cancer disparity in African American (AA) men.

The inventors disclose herein that deregulated PDEF expression could have profound effects on prostate cancer cell motility, invasiveness, and anchorage-independent growth and distant metastasis (FIGS. 1-4); as such loss of PDEF may be a critical determinant for acquisition of aggressiveness by prostate cancer. The inventors' data also discloses that that there is graded decrease in PDEF levels in prostate cancer cells with increasing aggressive phenotype and that PDEF is decreased in PCa cells derived from AA men. Moreover, similar decrease in PDEF expression was observed with in prostate cancer tissue sections of patients with high grade disease, and that PCa tissue sections from AA men show greater decrease in PDEF than those from grade matched Caucasian men (FIGS. 1, 5, and 6). As such PDEF could serve as a functional marker for distinguishing aggressive prostate cancer from an indolent disease in all men, and especially in AA men.

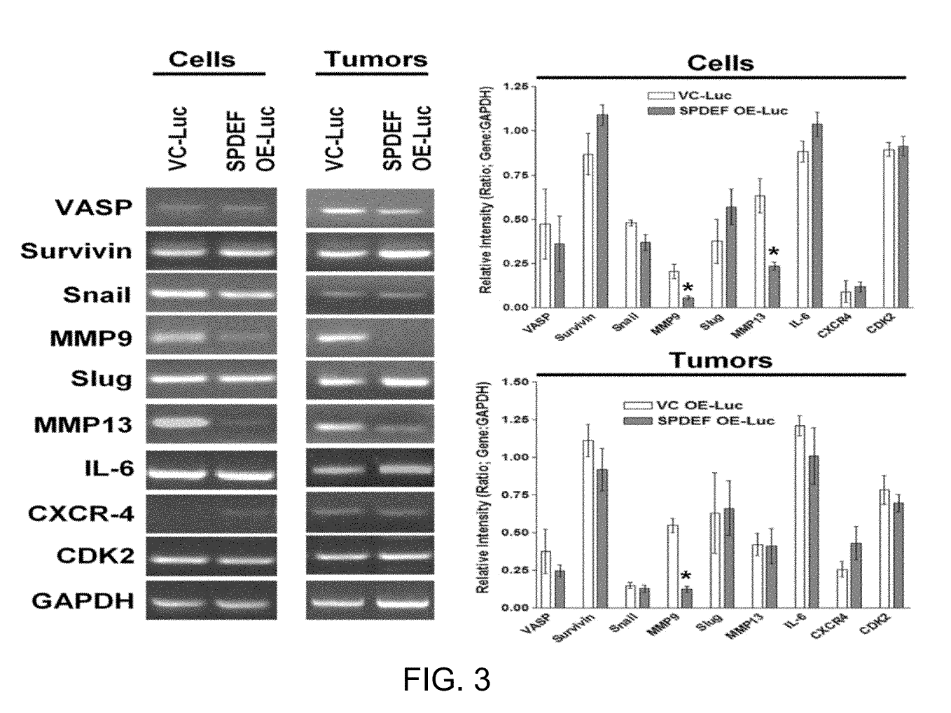

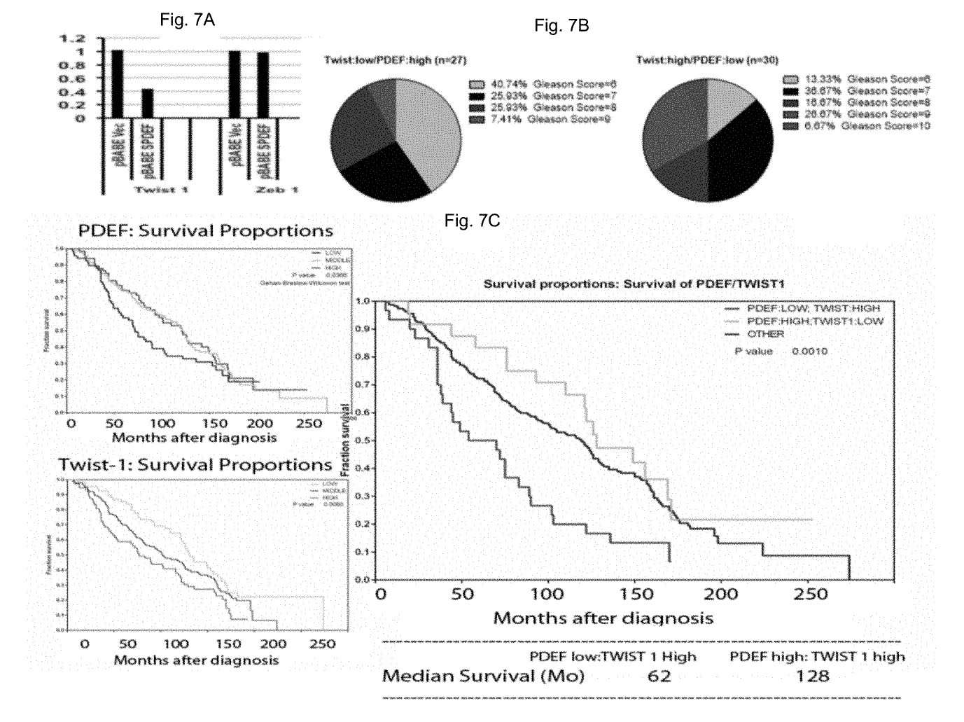

PDEF directly modulates gene expression by binding to one or more non-canonical Ets sites containing a GGAT core. PDEF is known to the inventors to alter expression of several genes that are also known Ets target genes. The inventors screened for expression of genes known to be regulated by PDEF in other cells following reintroduction of PDEF in PC3 cells. From this screen the inventors identified matrix metalloproteinase 9 (MMP 9) as one of the most down regulated genes following PDEF expression both in vitro and in vivo (FIG. 3). Disclosed herein, the inventors have identified Twist-1 as a new and novel target of PDEF (FIG. 7).

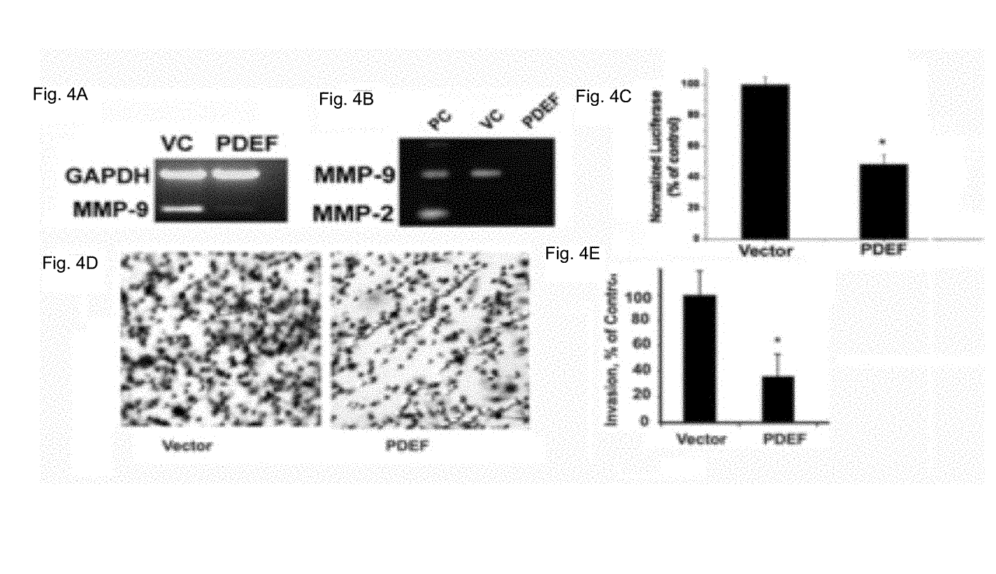

MMP9 belongs to a family of over 20 zinc-dependent enzymes that degrade various protein components of the extracellular matrix. MMPs play a critical role in tissue remodeling during development and tumor invasion, and metastasis in many tumors including prostate cancer. MMP9 is also of particular interest, because it is thought to be a tumor angiogenic factor that signals through the VEGFVEGFR system. Experimental overexpression of MMP-9 promotes pulmonary metastasis and inhibiting MMP-9 expression blocks tumor metastasis. In additional preliminary data the inventors confirmed that PDEF did indeed suppressed MMP9 enzymatic activity, MMP9 mRNA expression, as well as MMP9 promoter activity (FIG. 4). Moreover MMP9 expression in clinical samples of prostate cancer appeared to be inversely related to PDEF expression (not shown). These studies evidence that PDEF likely modulates tumor metastasis by regulating MMP9 activity.

TWIST1 protein is part of a large protein family of basic helix-loop-helix (bHLH) transcription factors. Each of these proteins includes a region called the bHLH domain, which determines the protein's 3-dimensional shape and enables it to target particular sequences of DNA. TWIST1 plays a key role in the regulation of embryogenesis and mesoderm formation during early embryonic development. TWIST1 plays a critical role in epithelial to mesenchymal transition (EMT). The inventors observed for the first time that over-expression of PDEF in prostate cancer cells resulted in decrease in Twist-1 mRNA levels. Moreover in clinical samples of prostate cancer decreased expression of PDEF and higher expression of Twist-1 was associated with high Gleason scores and predicted poor median survival from prostate cancer (FIG. 7).

The inventors disclose that PDEF expression may be used to predict risk of a non-invasive early-stage prostate cancer becoming metastatic, The inventors disclose the previously unrecognized role of PDEF in promoting metastases. The inventors disclose enabling patient stratification for selection of optimal therapy, which will particularly benefit the high-risk AA men and significantly reduce ethnic disparity in prostate cancer treatment outcomes. The inventors disclose a method to save patients with indolent prostate cancer from unnecessary treatments that compromise their quality of life. The inventors disclose a novel and unique causative link between PDEF and metastases. The inventors disclose a way for routine clinical use of PDEF as an independent predictor of metastases risk. Currently-used prognostic indicators (Digital rectal exam, PSA measurement and Gleason score) do not predict metastases risk accurately. Use of PDEF expression to distinguish between lethal (e.g., aggressive and metastatic in the short term) prostate cancer from an indolent disease (slow growing tumor an unlikely to be metastatic or be a cause of death) addresses an urgent, yet unmet in prostate cancer. Based on the data disclosed herein, the inventors propose using currently FDA approved DNA methylating drugs to halt prostate cancer metastasis.

Based on the disclosed data, it is evidenced that PDEF functions as a tumor metastasis suppressor in prostate cancer. Also based on the disclosed data, it is evidenced first, that in African American Men PDEF could serve as a functional marker for distinguishing aggressive prostate cancer from an indolent disease, second that PDEF functions as a metastasis suppressor in prostate cancer in part by modulating expression of target genes, and third, that loss of PDEF can help stratify PCa patients that might respond to DNA de-methylating agents,

PDEF expression is decreased in prostate cancer cells from AA men and in cells with aggressive phenotype, and experimental modulation of PDEF expression modulates cell migration and clonogenic activity, Evidence suggests that PDEF is a negative regulator of prostate tumor progression, including invasion and metastasis. in vitro, PDEF demonstrates differential expression in several prostate tumor cells lines via western blot (FIG. 1a). PDEF expression is inversely proportional to the aggressive nature of prostate tumor cell lines. Non-tumorigenic RWPE cells express PDEF (not shown), non-metastatic LNCaP cells express high amount of PDEF; whereas, PDEF protein cannot be detected in metastatic PC3 cells or in cell lines derived from PCa tissues from AA men (R77T and E006A). PDEF expression in C4-2B cells falls between LNCaP and PC3 cells, Therefore, to determine if PDEF plays a role in prostate tumor cell aggressiveness the inventors re-expressed PDEF in the PC3 cell line (FIG. 1b) and using shRNA technology, PDEF was knocked down in the LNCaP cell line (FIG. 1c), Using these cell lines, the inventors demonstrated that PDEF expression regulates tumor cell aggressiveness as demonstrated by decreased cell migration (FIG. 1d) and invasion (FIG. 1e) in the PDEF overexpressing PC3 cells, and increased cell migration (FIG. 1d) and invasion (FIG. 1e) in LNCaP cells expressing PDEF shRNA.

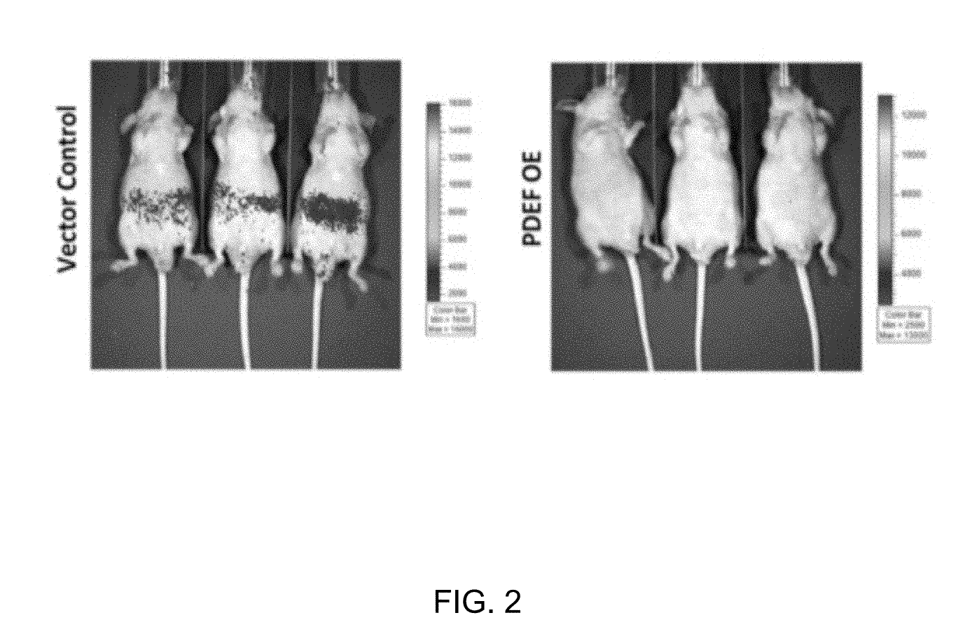

PDEF suppresses ability of prostate cancer cells to survive at secondary sites: Since the in vitro data suggested that PDEF expression was inversely associated with aggressive behavior of prostate cancer cells, the inventors initiated in vivo studies using PC3 cells with Luciferase construct (PC3-Luc) and then transfected these cells either with PDEF (SPDEF-Luc) or vector alone (VC-Luc). Next, these cells were used in two different mouse models of metastasis. The cells were either injected via the intra-cardiac route or via the tail vein (Not shown) to model hematogenous metastasis. As shown via bio-luminescence imaging in FIG. 2, PDEF expressing cells failed to successfully establish secondary metastasis (week 12 images). These data demonstrate that loss of PDEF is apparently a critical requirement for survival of prostate cancer cells at the secondary sites and provides the first direct demonstration of the role of PDEF in suppression of tumor metastasis in any system in vivo. It should be pointed out that the inventors' experimental metastasis model (tumor cell injection into intra-cardiac space) does not recapitulate all steps of the metastatic cascade; however, the use of this model allows the study of the survival of disseminated tumor cells in the circulation, extravasation, and growth at secondary sites.

PDEF specifically inhibited expression of Matrix metalloproteinase-9 (MMP-9) in prostate cancer cells and the effects of PDEF on cell invasion are mediated in part my MMP-9. The inventors screened for expression of a number of genes that have been shown to be regulated by PDEF in other cell systems and are associated with an aggressive phenotype. For these studies PC3 cells with or with our PDEF expression were either grown in vitro (tissue culture) or in vivo (xenograft) experiments. mRNA was isolated and gene expression was monitored by relative quantitative PCR. The results of these studies (FIG. 3) demonstrate that among the genes tested MMP9 was the only and most significantly down regulated genes both in vitro and in vivo.

The inventors also evaluated the effects of PDEF expression on MMP9 expression, promoter activity and enzymatic activity. Results show that PDEF expression completely abolished MMP-9 mRNA expression (FIG. 4A) and enzymatic activity (FIG. 4B), and significantly reduced MMP-9 promoter activity (FIG. 4C), as compared to vector control. These data disclose inhibition of MMP9 expression by PDEF, and disclose regulation of MMP9 by an ETS transcription factor. To test the effects of stable the ability of PC3 cells to invade simulated base-meat membrane in vitro, a phenotype correlated aggressive behavior. Results (FIGS. 4D and 4E) indicate that expression of PDEF significantly inhibited the ability of PC3 cells to invade through Matrigel compared with vector transfected control cells. Taken together these results demonstrate that PDEF negatively regulates MMP-9 expression and provides a potential mechanism by which PDEF could modulate invasive phenotype in prostate cancer.

Prostate Derived Ets Factor (PDEF) protein expression is decreased in clinical samples of high grade Human Prostate Cancer: The inventors evaluated PDEF expression in Tissue Microarray consisting of cores from normal and tumor regions. Results of these evaluations showed that PDEF is expressed in normal prostate tissue. However, in highgrade prostate cancer tissues, PDEF expression is decreased and even un-detectable in some areas of the tissue sections.

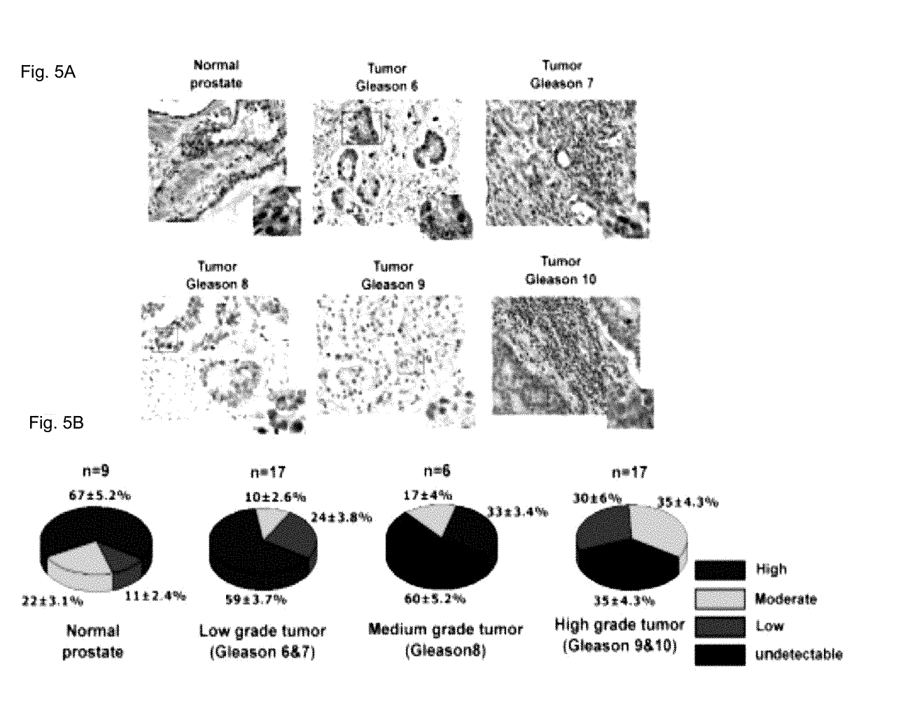

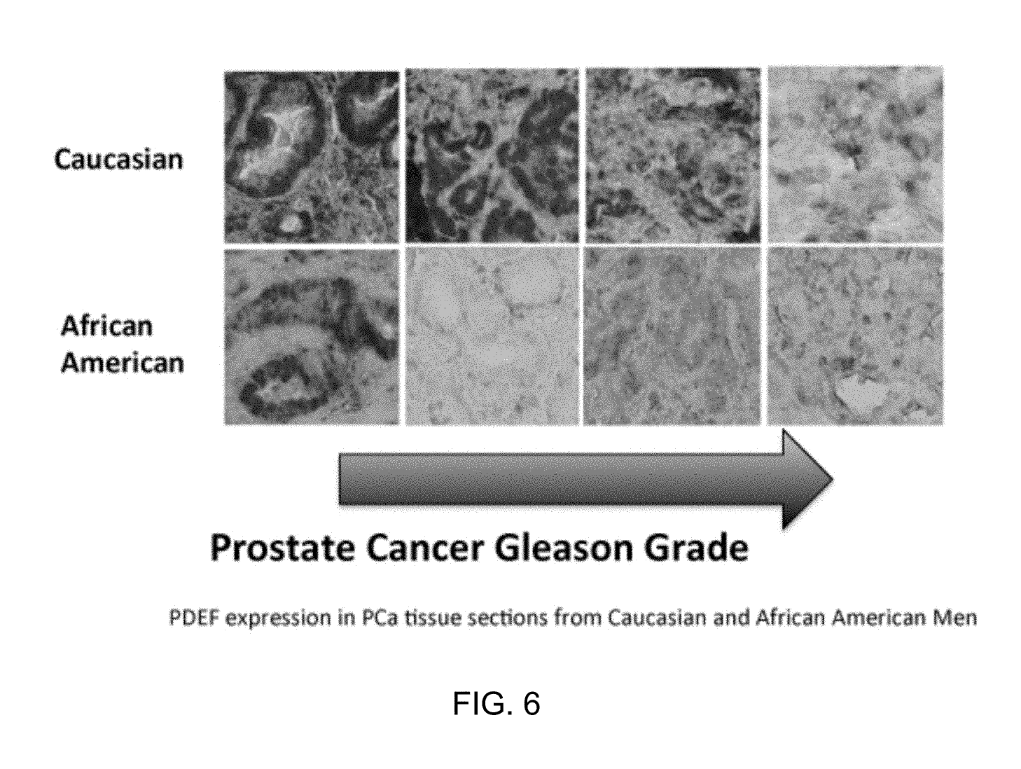

Interestingly, PDEF expression levels varied even within different areas of the same sections, indicating heterogeneous nature of prostate cancer. These data disclose an inverse relationship between PDEF expression and high Gleason grade for prostate cancer (FIG. 5). The inventors also analyzed prostate cancer sections on a tissue microarray (from CPCTR) with race specific annotation. Results (FIG. 6) show that there is a graded loss of PDEF in PCa sections with increasing Gleason score in both Caucasian and AA men. Moreover, there is disproportionate loss of PDEF in PCa samples from AA men. Moreover, tissue sections from AA men often showed complete loss of PDEF expression, even with a relatively lower Gleason grade.

PDEF expression is associated with decreased Twist-1 mRNA levels, and low PDEF/high TWIST-1 levels are predictors of aggressive PCa: The inventors observed that overexpression of PDEF in PC3 cells was associated with decrease in EMT marker, Twist-1 (FIG. 7A). The inventors also observed that low PDEF and high Twist-1 expression in clinical samples of PCa was associated with higher Gleason grade (FIG. 7B), and poor median survival (FIG. 7C). Patients with low PDEF had a median survival of 72 months compared to a median survival of 121 months for patients with high PDEF. Similarly patients with high TWIST 1 expression had a median survival of 77 months compared to a median survival of 131 months for patients with low TWIST 1. Moreover, when the inventors integrated expression of PDEF and TWIST 1 together, the inventors observed that patients with low PDEF and high TWIST 1 expression had a median survival of only 62 months compared to a median survival of 128 months for patients with high PDEF and Low TWIST 1. This data demonstrates usefulness of integrated expression of PDEF and TWIST 1 in separating aggressive PCa from an otherwise indolent disease. This data evidences that PDEF serves as a functional marker for distinguishing aggressive prostate cancer from an indolent disease in all men and especially African American men. This data further evidences that PDEF functions as a metastasis suppressor in prostate cancer in part by modulating expression of target genes.

AA men compared to Caucasian men continue to have the highest PCa incidence and death rates. Although with the advent of PSA screening early detection of PCa has increased, the disease is often well advanced in this population. There are currently no clinically used specific and selective biomarker/s that can accurately distinguish between aggressive PCa from an indolent disease. Thus, identifying biomarkers that may be used to identify those at particular risk of aggressive disease would influence PCa survival and other poor outcomes (Gleason score, stage at diagnosis and recurrence). The inventors show loss of PDEF in high-grade prostate cancer. The inventors' data also provide evidence that there is a disproportionate loss of PDEF in AA Men. In addition, the inventors also observed that MMP9 is one of the genes consistently down regulated by PDEF (FIGS. 4 and 5). Moreover, the inventors observed an inverse relationship between PDEF and MMP9 expression and PDEF and TWIST-1 expression (FIG. 7) in prostate cancer specimens.

The inventors data discloses that PDEF is lost in advanced prostate tumors, suggesting that PDEF has a suppressive effect on tumor metastasis (FIGS. 1 and 6). PDEF over-expression in PC3 cells decreases the motility and invasiveness in vitro (FIGS. 1 and 2), and PDEF overexpression in PC3 cells prevents the outgrowth of metastatic legions in an intra-cardiac injection tumor metastasis models (FIG. 3).

The inventors demonstrated in the disclosed data, PC3 cells have been engineered to over express PDEF (FIG. 1b).

Based upon the disclosed data, the inventors demonstrate that PDEF overexpression decreases metastatic burden compared to non-PDEF expressing vector control cells, as evidenced by decreased metastatic legions upon bio-luminescence imaging in two models of metastasis. Therefore, it is evidenced that similar results would be shown with PC3 and cell lines from AA men in the orthotopic model of tumor metastasis.

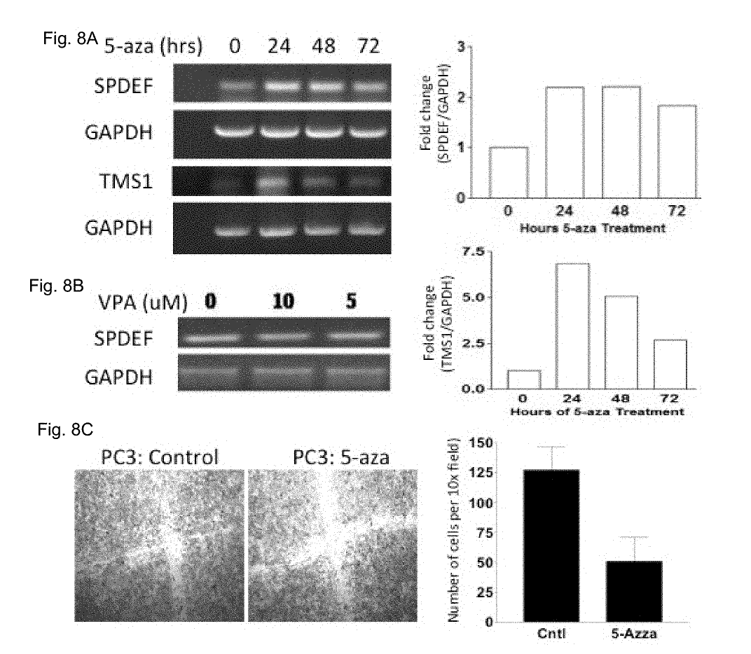

The inventors' data suggest that the expression of SPDEF is subjected to both epigenetic regulation (DNA methylation) and transcriptional modulation (FIG. 8-10). These data suggest that loss of PDEF can help stratify PCa patients that might respond to DNA de-methylating agents. That is, agents such as azacytidine and decitabine may modulate tumor progression and metastasis in prostate cancers with PDEF loss.

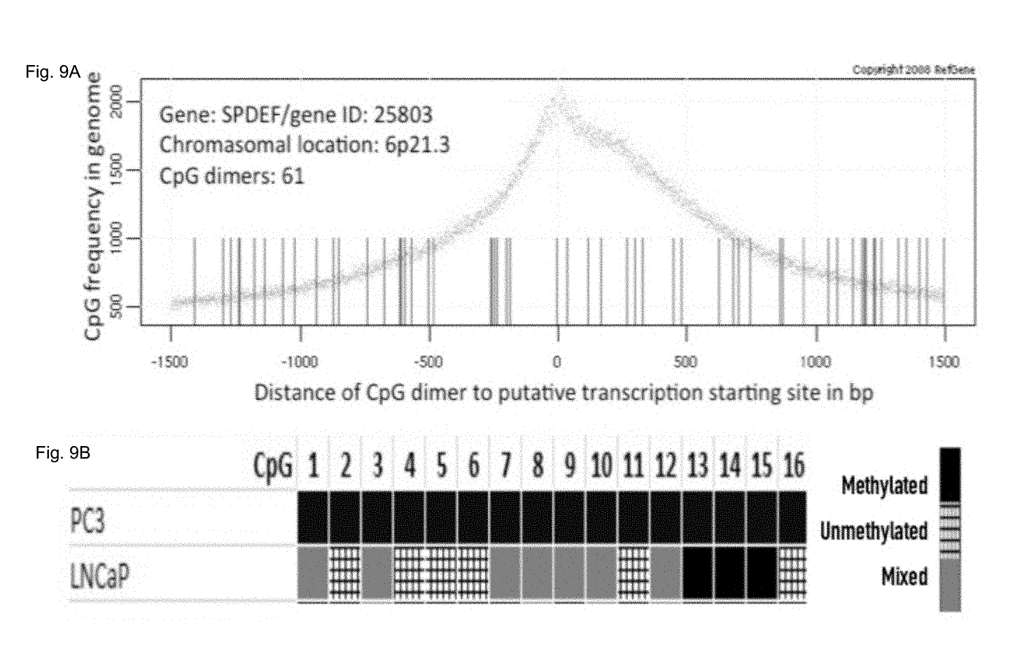

In addition to genetic alterations such as gene amplification and deletion, evidence suggests that epigenetic alterations also play roles in cancer disparity. Both culture and socioeconomic status can affect epigenetic processes; and the epigenetic modification effects can extend not only throughout the life course, but also to generations followed. DNA methylation and histone modifications are two common epigenetic mechanisms that control the expression of affected genes. The inventors found that PDEF is subjected to methylation regulation. As shown in FIG. 8, when PC3 cells were treated with DNA demethylation agent, 5-Aza, the expression of PDEF mRNA was induced. However, treatment with VPA, a HDAC inhibitor, did not alter the mRNA level of PDEF. Concomitant with the increased levels of SPDEF, 5-aza treatments reduced the cell migration and invasion ability of PC3 cells. Additionally, the inventors have also examined the distribution of CpG islands in the proximal promoter region of SPDEF. The inventors found that CpG islands were more frequently located at the transcription-starting site of PDEF gene (FIG. 9A) and that PDEF promoter was differentially methylated in PCa cells that have distinct metastatic ability (FIG. 9B). As shown in FIG. 9B, all the 16 CpG islands examined were methylated in the PC3 cells, which do not express detectable PDEF and are highly invasive and metastatic; whereas only 3 of the 16 CpG islands were methylated in LNCaP cells, which express PDEF and are less invasive and aggressive. Taken together, these data indicate that DNA methylation suppresses the expression of PDEF and loss of PDEF is closely associated with PCa aggressiveness. Expected treatment levels include, for example, azacytidine 2.5 mg/kg and decitabine 2.5 mg/kg.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, we claim:

1. A method of diagnosing a potentially lethal prostate cancer in a patient comprising:
measuring one of a level of SAM Pointed Domain Containing ETS Transcription Factor (SPDEF) expression in the patient, a level of TWIST1 protein in the patient, and a level of matrix metalloproteinase 9 (MMP-9) expression in the patient; and
diagnosing the patient with potentially lethal prostate cancer if one of the level of SPDEF in the patient is lower than a normal level of SPDEF, the level of TWIST1 in the patient is higher than a normal level of TWIST1, and the level of MMP-9 in the patient is higher than a normal level of MMP-9.

2. The method of claim 1 wherein the level of SPDEF expression in the patient is measured and the patient is diagnosed with potentially lethal prostate cancer if the level of SPDEF is lower than the normal level.

3. The method of claim 1 wherein the level of TWIST1 in the patient is measured and the patient is diagnosed with potentially lethal prostate cancer if the level of TWIST1 is higher than the normal level.

4. The method of claim 1 wherein the level of MMP-9 expression in the patient is measured and the patient is diagnosed with potentially lethal prostate cancer if the level of MMP-9 is higher than the normal level.

5. The method of claim 1 wherein both the level of SPDEF expression in the patient and the level of TWIST1 in the patient is measured and the patient is diagnosed with potentially lethal prostate cancer if both the level of SPDEF expression is lower than the normal level of SPDEF and the level of TWIST1 is higher than the normal level of TWIST1.

6. The method of claim 1 wherein both the level of SPDEF expression in the patient and the level of MMP-9 in the patient is measured and the patient is diagnosed with potentially lethal prostate cancer if both the level of SPDEF expression is lower than the normal level of SPDEF and the level of MMP-9 expression is higher than the normal level of MMP-9.

7. The method of claim 1 wherein both the level of TWIST1 in the patient and the level of MMP-9 in the patient is measured and the patient is diagnosed with potentially lethal prostate cancer if both the level of TWIST1 is higher than the normal level of TWIST1 and the level of MMP-9 expression is higher than the normal level of MMP-9.

8. The method of claim 1 wherein the level of SPDEF expression in the patient, the level of TWIST1 in the patient, and the level of MMP-9 expression in the patient is measured.

9. The method of claim 1 wherein the patient is diagnosed with potentially lethal prostate cancer if the level of SPDEF expression is lower than the normal level of SPDEF, the level of TWIST1 is higher than a normal level of TWIST1, and the level of MMP-9 expression is higher than the normal level of MMP-9.

10. The method of claim 1, wherein the normal level of SPDEF expression is lower if the patient is African American than if the patient is not African American.

11. A method of diagnosing and treating prostate cancer comprising:
measuring one of a level of SAM Pointed Domain Containing ETS Transcription Factor (SPDEF) expression in the patient, a level of TWIST1 protein in the patient, and a level of matrix metalloproteinase 9 (MMP-9) expression in the patient;
diagnosing the patient with potentially lethal prostate cancer if one of the level of SPDEF in the patient is lower than a normal level of SPDEF, the level of TWIST1 in the patient is higher than a normal level of TWIST1, and the level of MMP-9 in the patient is higher than a normal level of MMP-9, and
administering a therapeutic to the patient if diagnosed with potentially lethal prostate cancer.

12. The method of claim 11 wherein the therapeutic is a DNA demethylating agent.

13. The method of claim 11 wherein the therapeutic is one of azacytidine and decitabine.

14. The method of claim 11 wherein the therapeutic is SPDEF.

15. The method of claim 11 wherein the patient is human.

16. A kit for diagnosing potentially lethal prostate cancer in a patient comprising an assay for determining one of a level of SAM Pointed Domain Containing ETS Transcription Factor (SPDEF) expression in the patient, a level of TWIST1 protein in the patient, and a level of matrix metalloproteinase 9 (MMP-9) expression in the patient.
